(19) United States
(12) Patent Application Publication
(10) Pub. No.: US 2005/0215561 A1
(43) Pub. Date: Sep. 29, 2005

(54) Pharmaceutical dosage forms and compositions

(76) Inventors: Krishnendu Ghosh, Sparkill, NY (US); Arwinder S. Nagi, Thiells, NY (US); Xiaohong Pan, Highland Mills, NY (US); Melissa Lin, Wanaque, NY (US); Leonid Linberg, Upper Saddle River, NJ (US); Ping Cai, New City, NY (US); Eric N.C. Browne, Pierrefonds (CA); Michel Bernatchez, Montreal (CA); Mark Lankau, Dollard des Ormeaux (CA)

Correspondence Address: WOODCOCK WASHBURN LLP, ONE LIBERTY PLACE - 46TH FLOOR, PHILADELPHIA, PA, 19103, US

(21) Appl. No.: 11/082,548

(22) Filed: Mar. 17, 2005

Related U.S. Application Data
(60) Provisional application No. 60/554,622, filed on Mar. 19, 2004.

(51) Int. Cl. A61K 31/496; C07D 45/14

(52) U.S. Cl. 514/252.11; 544/357

(57) ABSTRACT

This invention relates to, for example, novel formulations and methods for the delivery of 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salts thereof, structurally related compounds and/or metabolites; as well as to use of these formulations and methods for treating disease.

### CROSS-REFERENCE TO RELATED APPLICATIONS

This application claims priority to provisional application Ser. No. 60/554,622 filed on Mar. 19, 2004 incorporated herein by reference in its entirety.

### FIELD

This invention relates, for example, to novel formulations and methods for the delivery of 4-cyano-N-{(2R)-2-[4-(2,3-dihydro-benzo[1,4]dioxin-5yl)-piperazin-1-yl ]-propyl}-N-pyridin-2-yl-benzamide, pharmaceutically acceptable salts thereof, structurally related compounds, and/or metabolites; as well as to use of these formulations and methods for treating disease.

### SUMMARY

The present invention provides, inter alia, formulations comprising 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salts thereof, structurally related compounds, metabolites, and combinations thereof.

Compounds provided by the present invention include 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof (e.g., 4-{(2R)-2-[4-(2,3-dihydro-benzo[1,4]dioxin-5yl)-piperazin-1-yl]-propyl}-N-pyridin-2-yl-benzamide hydrochloride salt); and structurally related compounds and metabolites thereof, including, but not limited to, {(2R)-2-[4-(2,3-dihydro-benzo[1,4]dioxin-5-yl)-piperazin-1-yl]-propyl}-N-pyridin-2-yl-amine or a pharmaceutically acceptable salt thereof; 4-cyano-N-{(2S)-2-[4-(2,3-dihydro-benzo[1,4]dioxin-5-yl)-piperazin-1-yl]-propyl}-N-pyridin-2-yl-benzamide or a pharmaceutically acceptable salt thereof; 4-cyano-N-(2-piperazin-1-yl-propyl)-N-pyridin-2-yl-benzamide or a pharmaceutically acceptable salt thereof; N-(5-chloro-pyridin-2-yl)-4-cyano-N-[2-(4-hydroxy-piperazin-1-yl)-propyl]-benzamide or a pharmaceutically acceptable salt thereof; N-(5-chloro-pyridin-2-yl)-4-cyano-N-{2-[4-(2,3-dihydro-benzo[1,4]dioxin-5-yl)-piperazin-1-yl]-propyl}-benzamide or a pharmaceutically acceptable salt thereof; 4-cyano-N-{(2R)-2-[4-(8-hydroxy-2,3-dihydro-benzo[1,4]dioxin-5-yl)-piperazin-1-yl]-propyl}-N-pyridin-2-yl-benzamide or a pharmaceutically acceptable salt thereof; 4-cyano-N-{(2R)-2-[4-(3-hydroxy-2,3-dihydro-benzo[1,4]dioxin-5-yl)-piperazin-1-yl]-propyl}-N-pyridin-2-yl-benzamide or a pharmaceutically acceptable salt thereof; 4-Cyano-N-{(2R)-2-[4-(2-hydroxy-2,3-dihydro-benzo[1,4]dioxin-5-yl)-piperazin-1-yl]-porpyl}-N-pyridin-2-yl-benzamide or a pharmaceutically acceptable salt thereof; 4-cyano-N-(2R-2-piperazin-1-yl-propyl)-N-pyridin-2-yl-benzamide or a pharmaceutically acceptable salt thereof; 4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]-2-methylpropyl}-2,3-dihydro-1,4-benzodioxin-5-yl)piperazin-1-yl]propyl}-N-pyridin-2-ylbenzamide or a pharmaceutically acceptable salt thereof; 4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]butyl}-2,3-dihydro-1,4-benzodioxin-5-yl)piperazin-1-yl]propyl}-N-pyridin-2-ylbenzamide or a pharmaceutically acceptable salt thereof; 4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]hexyl}-2,3-dihydro-1,4-benzodioxin-5-yl)piperazin-1-yl]propyl}-N-pyridin-2-ylbenzamide or a pharmaceutically acceptable salt thereof; 4-cyano-N-{(2R)-2-[4-(8-{[8-(4-{(1S)-2-[(4-cyanobenzoyl)(pyridine-2-yl)amino]-1-methylethyl}piperazin-1-yl)-2,3-dihydro-1,2-benzodioxin-5-yl]methyl}-2,3-dihydro-1,4-benzodioxin-5-yl)piperazin-1-yl]propyl}-N-pyridin-2-ylbenzamide or a pharmaceutically acceptable salt thereof; 4-cyano-N-{(2R)-2-[4-(8-{1-[4-{(1S)-2-[(4-cyanobenzoyl)(pyridine-2-yl)amino]-1-methylethyl}piperazin-1-yl)-2,3-dihydro-1,4-benzodioxin-5-yl]ethyl}-2,3-dihydro-1,4-benzodioxin-5-yl)piperazin-1-yl]propyl}-N-pyridin-2-ylbenzamide or a pharmaceutically acceptable salt thereof; and 4-cyano-N-[2(R)-(4-cyano-benzamido)-propyl]-N-pyridin-2-yl-benzamide or a pharmaceutically acceptable salt thereof.

In one embodiment, the compounds are in the form of particles. In one aspect, the particles will have a mean diameter of no more than about 20 microns. In another aspect, the particles will have a mean diameter of from about 0.75 to about 10 microns. In another aspect, the particles will have a mean diameter of from about 2 to about 8 microns.

Compositions of the present invention comprise 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof (e.g., 4-{(2R)-[4-(2,3-dihydro-benzo[1,4]dioxin-5yl)-piperazin-1-yl]-propyl}-N-pyridin-2-yl-benzamide hydrochloride salt), structurally related compounds or metabolites thereof as described herein. In some embodiments, compositions of the present invention will comprise 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof and one or more structurally related compounds and/or metabolites. In some embodiments, 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof and its structurally related compounds and/or metabolites will be present in the composition in the form of particles. In one aspect, the particles will have a mean diameter of no more than about 20 microns. In another aspect, the particles will have a mean diameter of from 0.75 to about 10 microns. In another aspect, the particles will have a mean diameter of from about 2 to about 8 microns. In some embodiments, the structurally related compounds and/or metabolites when provided in a composition with 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof (e.g., 4-{(2R)-2-[4-(2,3-dihydro-benzo[1,4]dioxin-5yl)-piperazin-1-yl]-propyl}-N-pyridin-2-yl-benzamide hydrochloride salt) will be in an amount of less than about 0.1 weight percent each. In some embodiments, compositions of the present invention will further comprise a pharmaceutically acceptable carrier.

In some embodiments, compositions and dosage forms of the present invention comprising 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof will be substantially free of one or more dimers of 4-cyano-N-{(2R)-2-[4-(2,3-dihydro-benzo[1,4]dioxin-5yl)-piperazin-1-yl]-propyl}-N-pyridin-2-yl-benzamide. Substantially free, as used in this context, means that the dimers will be present in the compositions in an amount of less than about 0.5 weight percent each, preferably in an amount of less than about 0.3 weight percent each, more preferably in an amount of less than about 0.2 weight percent each, and even more preferably in an amount of less than about 0.1 weight percent each, based on the total weight of the composition, and in the dosage forms in an amount of less than about 0.5 weight percent each, preferably in an amount of less than about 0.3 weight percent each, more preferably in an amount of less than about 0.2 weight percent each, and even more preferably in an amount of less than about 0.1 weight percent each, based on the weight of the active ingredient in the dosage form. Accordingly, the present invention provides formulations comprising 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof that are substantially free of dimers of 4-cyano-N-{(2R)-2-[4-(2,3-dihydro-benzo[1,4]dioxin-5yl)-piperazin-1-yl]-propyl}-N-pyridin-2-yl-benzamide and/or other structurally related compounds of 4-cyano-N-{(2R)-2-[4-(2,3-dihydro-benzo[1,4]dioxin-5yl)-piperazin-1-yl]-propyl}-N-pyridin-2-yl-benzamide. Representative dimers of 4-cyano-N-{(2R)-2-[4-(2,3-dihydro-benzo[1,4]dioxin-5yl)-piperazin-1-yl]-propyl}-N-pyridin-2-yl-benzamide are as shown in formulas 7 and 8.

Dosage forms of the present invention comprise 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof (e.g., 4-{(2R)-2-[4-(2,3-dihydro-benzo[1,4]dioxin-5yl)-piperazin-1-yl]-propyl}-N-pyridin-2-yl-benzamide hydrochloride salt), structurally related compounds or metabolites as described herein. In some embodiments, dosage forms of the present invention will comprise 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof and one or more structurally related compounds and/or metabolites. In some embodiments, 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof and its structurally related compounds and/or metabolites will be present in the dosage form in the form of particles. In one aspect, the particles will have a mean diameter of no more than about 20 microns. In another aspect, the particles will have a mean diameter of from 0.75 to about 10 microns. In another aspect, the particles will have a mean diameter of from about 2 to about 8 microns. In some embodiments, the structurally related compounds and/or metabolites when provided in a dosage form with 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof (e.g., 4-{(2R)-2-[4-(2,3-dihydro-benzo[1,4]dioxin-5yl)-piperazin-1-yl]-propyl}-N-pyridin-2-yl-benzamide hydrochloride salt) will be in an amount of less than about 0.1 weight percent each.

In some embodiments, the active ingredient(s) in a dosage form of the present invention is effective to achieve a maximal plasma concentration about 1 to about 12 hours following administration. In one aspect, the active ingredient(s) in a dosage form of the present invention will be effective to achieve a maximal plasma concentration about 1 to about 4 hours following administration.

In some embodiments, the active ingredient is released at a rate that is effective to achieve a plasma concentration that is about 50% of the maximal plasma concentration at about 15 hours following administration, preferably the active ingredient will be released at a rate that is effective to achieve a plasma concentration that is about 50% of the maximal plasma concentration at about 1 to about 10 hours following administration.

The term active ingredient refers to 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof (e.g., 4-{(2R)-2-[4-(2,3-dihydro-benzo[1,4]dioxin-5yl)-piperazin-1-yl]-propyl}-N-pyridin-2-yl-benzamide hydrochloride salt), structurally related compounds or metabolites (as shown herein) and their pharmaceutically acceptable salts.

In some embodiments, pharmaceutical compositions and/or dosage forms comprise in addition to the active ingredient (e.g., 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof) at least one rate controlling polymer and at least one organic acid. In some embodiments, the organic acid is citric acid anyhydrate, citric acid monohydrate, ascorbic acid, aspartic acid, glutamic acid, fumaric acid, malic acid or tartaric acid. In some embodiments, the organic acid is citric acid or a polyfunctional organic acid. In some embodiments, the at least one release rate controlling polymer is a methylcellulose. In some embodiments, the polymer is a hydroxypropyl methylcellulose, hydroxypropyl cellulose, hydroxyethyl cellulose or hydroxypropyl methylcellulose phthalate. In some embodiments, the hydroxypropyl methylcellulose is hypromellose 2208 or 2910 (e.g., Methocel™ K4M, Methocel™ K15M, Methocel™ KI00M, Methocel™ E10M, Methocel™ E4M, Methocel™ K100LV, Methocel™ E50LV, Methocel™E5, Methocel™ E6, or Methocel™ E15LV. In some embodiments, the organic acid is citric acid and the rate controlling polymer is hypromellose 2208, (e.g., Methocel™ K4M premium CR and/or Methocel™ K100M Premium CR).

In some embodiments, pharmaceutical compositions and/or dosage forms further comprise at least one filler. In some embodiments, the filler is microcrystalline cellulose, lactose, calcium carbonate, calcium phosphate, maltodextrin, dextrose, fructose, maltose, mannitol, starch, or sucrose. In some embodiments, the microcrystalline cellulose is silicified microcrystalline cellulose and the lactose is lactose monohydrate. In some embodiments, pharmaceutical compositions and/or dosage forms further comprise at least one lubricant. In some embodiments, the lubricant is magnesium stearate, talc, stearic acid, or colloidal silicon dioxide. Accordingly in some embodiments, pharmaceutical compositions and/or dosage forms of the present invention comprise, in addition to the active ingredient or ingredients, at least one rate controlling polymer, at least one organic acid, at least one filler, and at least one lubricant.

In some embodiments, pharmaceutical compositions and/or dosage forms of the present invention comprise about 2 to about 45 or 46 parts of a release rate controlling polymer and about 1 to about 5 parts of an organic acid per part of active ingredient. In some embodiments, the pharmaceutical compositions and/or dosage forms comprise about 0.4 to about 10 mg of active ingredient. In some embodiments, the pharmaceutical compositions and/or dosage forms of the present invention comprise about 50 to about 150 mg of rate controlling polymer(s), about 5 to about 50 mg of organic acid(s), about 85 to about 179 mg of filler(s) and about 1 mg of lubricant. In some embodiments, there will be from about 2 to about 50 mg of organic acid(s).

In some embodiments, pharmaceutical compositions and/or dosage forms of the present invention comprise in addition to the active ingredient (e.g., 4-cyano-N-{(2R)-[4-(2,3-dihydro-benzo[1,4]dioxin-5yl)-piperazin-1-yl]-propyl}-N-pyridin-2-yl-benzamide or a pharmaceutically acceptable salt form thereof), at least one filler and at least one lubricant. In some embodiments, the filler is microcrystalline cellulose, lactose, calcium carbonate, calcium phosphate, maltodextrin, dextrose, fructose, maltose, mannitol, starch, sucrose or a blend thereof. In some embodiments, the filler is microcrystalline cellulose, lactose, or a blend thereof. In some embodiments, pharmaceutical compositions and/or dosage forms further comprise at least one lubricant. In some embodiments, the lubricant is magnesium stearate, talc, stearic acid, or colloidal silicon dioxide. In some embodiments, the lubricant is magnesium stearate.

In some embodiments, pharmaceutical compositions and/or dosage forms comprise about 15 to about 300 parts of filler and about 0.1 to about 3 parts of lubricant per part of active ingredient. In some embodiments, the pharmaceutical compositions and/or dosage forms comprise about 0.1 to about 5 mg of 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt form thereof. In some embodiments, the pharmaceutical compositions and/or dosage forms comprise about 80 to about 150 mg of one or more filler(s) and at least about 0.75 mg of one or more lubricant(s).

In some embodiments, the dosage forms of the present invention are in the form of tablets. In one aspect, the tablets are film coated.

In some embodiments, the compositions or dosage forms of the present invention are in the form of a dry blend.

The present invention provides processes of providing the compositions and dosage forms of the present invention. In some embodiments, the compositions are compressed for a time and under conditions effective to form a tablet thereof. In some embodiments, the tablets are further film coated.

The present invention also provides processes comprising mixing the active ingredient, at least one rate controlling polymer and at least one organic acid thereby forming a blend thereof. In some embodiments, the process further comprises compressing the blend for a time and under conditions effective to form a tablet thereof. In some embodiments, the tablets are further film coated.

The present invention also provides processes comprising mixing the active ingredient, at least one filler and at least lubricant thereby forming a blend thereof. In some embodiments, the process further comprises compressing the blend for a time and under conditions effective to form a tablet thereof. In some embodiments, the tablets are further film coated.

In some embodiments, the dosage forms of the present invention are free of base.

In some embodiments, the present invention provides methods and processes of administering a dosage form, compound or composition of the present invention to a mammal, e.g., to a human. In some embodiments, the dosage forms, compounds or compositions are orally administered. In one aspect, they are orally administered once every 12 or 24 hours. In another aspect, they are orally administered once every 48 hours. In some particularly preferred embodiments, the dosage forms, compounds or compositions are administered to treat Alzheimer's Disease.

### DETAILED DESCRIPTION

The present invention provides, inter alia, formulations comprising 4-cyano-N-{(2R)-2-[4-(2,3-dihydro-benzo[1,4]dioxin-5yl)-piperazin-1-yl]-propyl}-N-pyridin-2-benzamide, pharmaceutically acceptable salts thereof, structurally related compounds, and/or metabolites. As used herein, the term “formulations” refers to compounds, compositions, and dosage forms, such as, for example, immediate release and sustained release dosage forms.

The present invention also provides processes for making the formulations and methods of administering them to a mammal.

Preferred formulations for use in the present invention are those that act as serotonergic agents and have 5-HT_{1A} binding activity. In particular, preferred compounds act as 5-HT_{1A} antagonists. See, for example, U.S. Pat. Nos. 6,784,294, 6,713,626, 6,469,007, 6,586,436, 5,710,149, and 6,127,357, and WO 97/03982, the disclosures of which are incorporated herein by reference in their entirety for all purposes. Compounds of the present invention, as well as compositions comprising more than one compound of the present invention, can be prepared by those skilled in the art of organic synthesis employing known methods that utilize readily available reagents and starting materials, see, for example, EP0512755 B1, WO 97/03982, U.S. Pat. Nos. 6,127,357, 6,469,007, 6,713,626, and 6,784,294, and U.S. Published Application No. 20030208075A1, the disclosures of which are incorporated herein by reference in their entirety for all purposes.

Such methods include alkylating 1-(2,3-dihydro-1,4-benzodioxin-5-yl)piperazine hydrochloride with sulfamate 4,5-dihydro-5S-methyl-3-(2-pyridinyl)-3H[1.2.3]oxathiazole-2,2-dioxide to give a sulfamic acid intermediate which is hydrolyzed to{(2R)-2-[4-(2,3-dihydro-benzo[1,4]dioxin-5yl)-piperazin-1-yl]-propyl}pyridin-2-yl-amine and then treating {(2R)-2-[4-(2,3-dihydro-benzo[1,4]dioxin-5yl)-piperazin-1-yl]-propyl}pyridin-2-yl-amine with 4-cyanobenzoyl chloride to give 4-cyano-N-{(2R)-2-[4-(2,3-dihydro-benzo[1,4]dioxin-5yl)-piperazin-1-yl]-propyl}-N-pyridin-2-yl-benzamide base. Treatment of 4-cyano-N-{(2R)-2-[4-(2,3-dihydro-benzo[1,4]dioxin-5yl)-piperazin-1-yl]-propyl}-N-pyridin-2-yl-benzamide base with hydrochloric acid gives its hydrochloride salt.

In some embodiments of the present invention, preparations comprising 4-cyano-N-{(2R)-2-[4-(2,3-dihydro-benzo[1,4]dioxin-5yl)-piperazin-1-yl]-propyl}-N-pyridin-2-yl-benzamide and its pharmaceutically acceptable salts are further processed and purified. For example, in one embodiment, a preparation comprising p4-cyano-N-{(2R)-2-[4-(2,3-dihydro-benzo[1,4]dioxin-5yl)-piperazin-1-yl]-propyl}-N-pyridin-2-yl-benzamide prepared by methods disclosed herein is dissolved in organic solvent, treated with silica gel, and filtered in order to remove structurally related compounds, e.g., dimers represented by Formulas 7 and 8. The remaining product can then be concentrated and re-crystallized in order to provide, e.g., 4-cyano-N-{(2R)-2-[4-(2,3-dihydro-benzo[1,4]dioxin-5yl)-piperazin-1-yl]-propyl}-N-pyridin-2-yl-benzamide hydrochloride salt.

Preferred formulations of the present invention can be used to modulate, e.g., antagonize or agonize, 5-HT_{1A} receptor activity and are useful in the treatment of diseases such as CNS disorders, including, but not limited to, schizophrenia, (and other psychotic disorders such as paranoia and mano-depressive illness), Parkinson's disease and other motor disorders, anxiety (e.g., generalized anxiety disorders, panic attacks, and obsessive compulsive disorders), depression (such as by the potentiation of serotonin reuptake inhibitors and serotonin norepinephrine reuptake inhibitors), Alzheimer's disease, Tourette's syndrome, migraine, autism, attention deficit disorders and hyperactivity disorders. Preferred formulations are useful for the treatment of sleep disorders, social phobias, pain, thermoregulatory disorders, endocrine disorders, urinary incontinence, vasospasm, stroke, eating disorders such as for example obesity, anorexia and bulimia, sexual dysfunction, and the treatment of alcohol, drug and nicotine withdrawal.

Preferred formulations of the present invention are also useful for the treatment of cognitive dysfunction including but not limited to cognitive dysfunction associated with mild cognitive impairment (MCI), Alzheimer's disease and other dementias including Lewy Body, vascular, and post stroke dementias. Cognitive dysfunction associated with surgical procedures, traumatic brain injury or stroke can also be treated in accordance with the present invention. Further, preferred formulations are useful for the treatment of diseases in which cognitive dysfunction is a co-morbidity such as, for example, Parkinson's disease, autism and attention deficit disorders.

Despite its high solubility in water (about 51 mg/ml at 25° C.), 4-cyano-N-{(2R)-2-[4-(2,3-dihydro-benzo[1,4]dioxin-5yl)-piperazin-1-yl]-propyl}-N-pyridin-2-yl-benzamide and its salts are preferably provided in micronized form. As such, the present invention provides formulations comprising 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salts thereof, structurally related compounds, or metabolites in micronized and in non-micronized form. For purposes of the present invention, a compound in micronized form is in the form of particles having a mean diameter of no more than about 20 microns. It will be understood that compounds of the present invention can be in the form of particles having a mean diameter of greater than about 20 microns, for example in the form of particles having a mean diameter from about 20 microns to about 300 or about 500 microns. Preferably, the particles have a mean diameter of about 10 microns, more preferably a mean diameter from about 0.75 to about 10 microns, even preferably from about 2 to about 8 microns. Methods of micronization or particle size reduction are known and are thus not described herein in detail.

As will be recognized, 4-cyano-N-{(2R)-2-[4-(2,3-dihydro-benzo[1,4]dioxin-5yl)-piperazin-1-yl]-propyl}-N-pyridin-2-yl-benzamide is represented by the following formula:

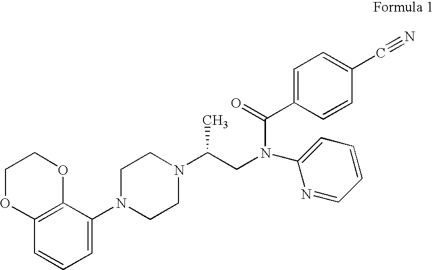

Within the present invention, the compounds of formula 1 can be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include, for example, inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, suluric, tartaric acid, p-toluenesulfonic and the like. Particularly preferred are hydrochloric, hydrobromic, phosphoric, and sulfuric acids, and most preferably is the hydrochloride salt.

In certain embodiments, formulations comprising 4-cyano-N-{(2R)-2-[4-(2,3-dihydro-benzo[1,4]dioxin-5yl)-piperazin-1-yl]-propyl}-N-pyridin-2-yl-benzamide or pharmaceutically acceptable salts thereof will also comprise one or more structurally related compounds that can be detected and quantified using known methods. Examples of such structurally related compounds include, but are not limited to, those compounds represented by Formulas 2-9 and pharmaceutically acceptable salts thereof, including, for example, {(2R)-2-[4-(2,3-dihydro-benzo[1,4]dioxin-5-yl)-piperazin-1-yl]-propyl}-N-pyridin-2-yl-amine or a pharmaceutically acceptable salt thereof; 4-cyano-N-{(2S)-2-[4-(2,3-dihydro-benzo[1,4]dioxin-5-yl)-piperazin-1-yl]-propyl}-N-pyridin-2-yl-benzamide or a pharmaceutically acceptable salt thereof; 4-cyano-N-(2-piperazin-1-yl-propyl)-N-pyridin-2-yl-benzamide or a pharmaceutically acceptable salt thereof; 4-cyano-N-[(2R)-2-piperazin-1-yl-propyl]-N-pyridin-2-yl-benzamide or a pharmaceutically acceptable salt thereof; N-(5-chloro-pyridin-2-yl)-4-cyano-N-[2-(4-hydroxy-piperazin-1-yl)-propyl]-benzamide or a pharmaceutically acceptable salt thereof; N-(5-chloro-pyridin-2-yl)-4-cyano-N-[(2R)-2-(4-hydroxy-piperazin-1-yl)-propyl]-benzamide or a pharmaceutically acceptable salt thereof; N-(5-chloro-pyridin-2-yl)-4-cyano-N-{2-[4-(2,3-dihydro-benzo[1,4]dioxin-5-yl)-piperazin-1-yl]-propyl}-benzamide or a pharmaceutically acceptable salt thereof; N-(5-chloro-pyridin-2-yl)-4-cyano-N-{(2R)-2-[4-(2,3-dihydro-1,4-benzodioxin-5-yl)-piperazin-1-yl]-propyl}benzamide or a pharmaceutically acceptable salt thereof; 4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]-2-methylpropyl}-2,3-dihydro-1,4-benzodioxin-5-yl)piperazin-1-yl]propyl}-N-pyridin-2-ylbenzamide or a pharmaceutically acceptable salt thereof; 4-cyano-N-{(2R)-2-[4-(8-{1-[8-(4-{(1S)-2-[(4-cyclobenzoyl)(pyridine-2-yl)amino]-1-methylethyl}piperazin-1-yl)-2,3-dihydro-1,4-benzodioxin-5-yl]butyl}-2,3-dihydro-1,4-benzodioxin-5-yl)piperazin-1-yl]propyl}-N-pyridin-2-ylbenzamide or a pharmaceutically acceptable salt thereof; 4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]hexyl}-2,3-dihydro-1,4-benzodioxin-5-yl)piperazin-1-yl]propyl}-N-pyridin-2-ylbenzarnide or a pharmaceutically acceptable salt thereof; 4-cyano-N-{(2R)-2-[4-(8-{[8-(4-{(1S)-2-[(4-cyanobenzoyl)(pyridine-2-yl)amino]-1-methylethyl}piperazin-1-yl)-2,3-dihydro-1,4-benzodioxin-5-yl]methyl}-2,3-dihydro-1,4-benzodioxin-5-yl)piperazin-1-yl]propyl}-N-pyridin-2-ylbenzamide or a pharmaceutically acceptable salt thereof; 4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]ethyl}-2,3-dihydro-1,4-benzodioxin-5-yl)piperazin-1-yl]propyl}-N-pyridin-2-ylbenzamide or a pharmaceutically acceptable salt thereof; and 4-cyano-N-[2(R)-(4-cyano-benzamido)-propyl]-N-pyridin-2-yl-benzamide or a pharmaceutically acceptable salt thereof. 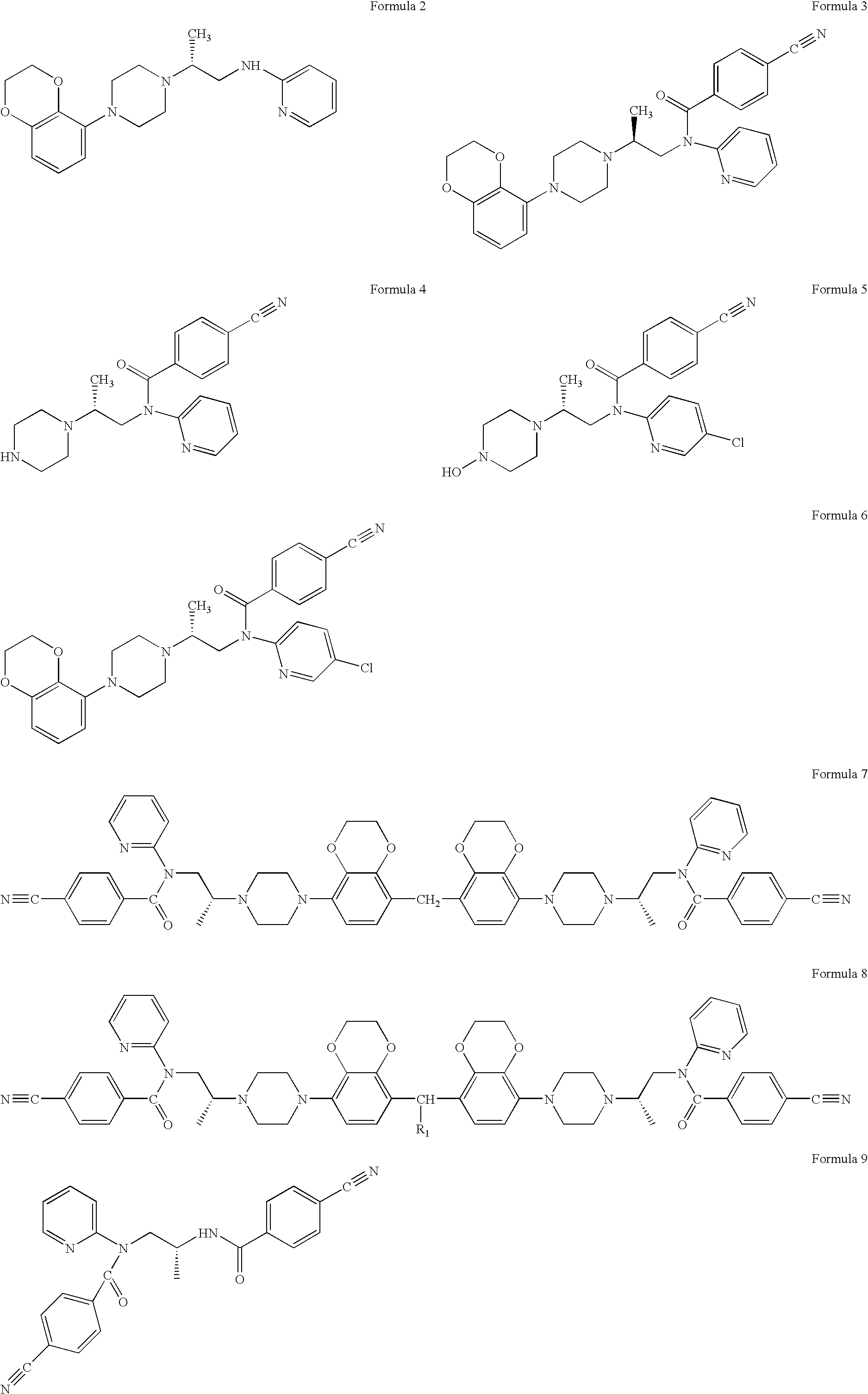
wherein R_{‘}is —CH₃,—-CH(CH₃)₂, —CH₂CH₂CH₃, CH₂CH₂CH₂CH₃ or —CH₂CH₂CH₂CH₂CH₃.

In some embodiments, the present invention provides formulations comprising one or more compounds represented by Formulas 2, 3, 4, 5, 6, 7, 8 or 9 or a pharmaceutically acceptable salt thereof. In some aspects of the present invention, the formulations will comprise 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt thereof and one or more compounds of Formula 2, 3, 4, 5, 6, 7, 8 or 9 or a pharmaceutically acceptable salt thereof. In some embodiments, for example, formulations of the present invention can comprise 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt thereof, {(2R)-2-[4-(2,3-dihydro-benzo[1,4]dioxin-5-yl)-piperazin-1-yl]-propyl}-N-pyridin-2-yl-amine or a pharmaceutically acceptable salt thereof, and 4-cyano-N-{(2S)-2-[4-(2,3-dihydro-benzo[1,4]dioxin-5-yl)-piperazin-1-yl]-propyl}-N-pyridin-2-yl-benzamide or a pharmaceutically acceptable salt thereof. Accordingly, the present invention provides formulations comprising 4-cyano-N-{(2R)-2-[4-(2,3-dihydro-benzo[1,4]dioxin-5yl)-piperazin-1-yl]-propyl}-N-pyridin-2-yl-benzamide hydrochloride salt, {(2R)-2-[4-(2,3-dihydro-benzo[1,4]dioxin-5-yl)-piperazin-1-yl]-propyl}-N-pyridin-2-yl-amine, and 4-cyano-N-{(2S)-2-[4-(2,3-dihydro-benzo[1,4]dioxin-5-yl)-piperazin-1-yl]-propyl}-N-pyridin-2-yl-benzamide hydrochloride salt. When the structurally related compounds described above are present in combination with 4-cyano-N-{(2R)-2-[4-(2,3-dihydro-benzo[1,4]dioxin-5yl)-piperazin-1-yl]-propyl}-N-pyridin-2-yl-benzamide or a pharmaceutically acceptable salt thereof, the former preferably predominates and the latter preferably are present in the composition in amount of less than about 10%, more preferably present in amount of less than about 5% and even more preferably in amounts of less than about 1% or 0.1%, for example, in amounts from between about 0.08% and about 0.27%.

4-Cyano-N-{2-[4-(2,3-dihydro-benzo[1,4]dioxin-5yl)-piperazin-1-yl]-propyl}-N-pyridin-2-yl-benzamide contains one chiral center and is used predominately as the R-isomer. The formulations, e.g., compounds, compositions, or dosage forms, of the present invention can include both R and S isomers, and are not limited to a single enantiomer or particular enantiomeric mixture.

The present invention also provides formulations comprising metabolites of 4-cyano-N-{(2R)-2-[4-(2,3-dihydro-benzo[1,4]dioxin-5yl)-piperazin-1-yl]-propyl}-N-pyridin-2-yl-benzamide. Metabolites include, but are not limited to, 4-cyano-N-{(2R)-2-[4-(8-hydroxy-2,3-dihydro-benzo[1,4]dioxin-5-yl)-piperazin-1-yl]-propyl}-N-pyridin-2-yl-benzamide or a pharmaceutically acceptable salt thereof, 4-cyano-N-{(2R)-2-[4-(3-hydroxy-2,3-dihydro-benzo[1,4]dioxin-5-yl)-piperazin-1-yl]-propyl}-N-pyridin-2-yl-benzamide or a pharmaceutically acceptable salt thereof, 4-Cyano-N-{(2R)-2-[4-(2-hydroxy-2,3-dihydro-benzo[1,4]dioxin-5-yl)-piperazin-1-yl]-propyl}-N-pyridin-2-yl-benzamide or a pharmaceutically acceptable salt thereof, and 4-cyano-N-(2R-2-piperazin-1-yl-propyl)-N-pyridin-2-yl-benzamide or a pharmaceutically acceptable salt thereof. As will be recognized, these metabolites are represented by Formulas 10-13. It will be recognized that these metabolites can be employed as pharmaceutically active compounds and in pharmaceutical dosage forms in their own right, alone or in combination with other pharmaceutically active compounds.

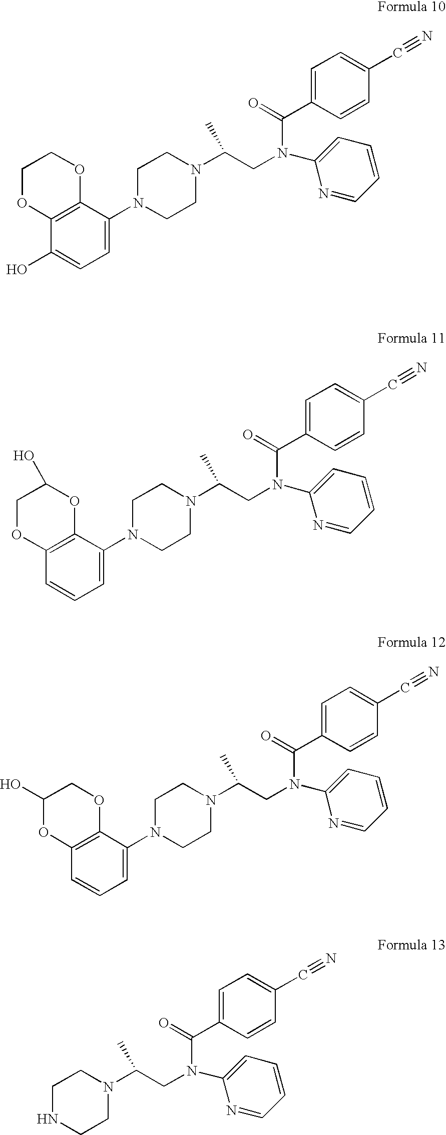

The present invention provides immediate release and sustained release dosage forms comprising one or more active ingredients, e.g., 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salts thereof, structurally related compounds and metabolites thereof that have 5-HT_{1A} binding activity.

A drug “release rate” refers to the quantity of drug released from a dosage form per unit time, e.g., milligrams of drug released per hour (mg/hr). Drug release rates can be calculated, for example, under in vitro dosage form dissolution testing conditions known in the art. As used herein, a drug release rate obtained at a specified time “following administration” refers to the in vitro drug release rate obtained at the specified time following implementation of an appropriate dissolution test. Methods of performing dissolution tests or release rate assays are known in the art. The time at which a specified percentage of the drug within a dosage form has been released can be referenced as the “Tₓ” value, where “x” is the percent of drug that has been released. A commonly-used reference measurement for evaluating drug release from oral dosage forms is the time at which 70% or 90% of drug within a dosage form has been released. This measurement is referred to as “T₇₀” or “T₉₀” for the dosage form.

For purposes of this invention, the terms “immediate release formulation” refer to formulations that provide a relatively rapid and non-gradual release of active compound from the formulation; e.g., formulations that contain active compound and a rapidly dissolving carrier that does not retard the release of the active compound from the formulation. Such immediate release formulation are either devoid of release rate controlling polymers or other species that retard the release of the active compound from the formulation, or contain such polymers or species in amounts that are sufficiently small such that the release of the active compound from the formulation is not retarded relative to an otherwise identical formulation lacking such polymers or species. One example of such an immediate release formulation is the active ingredient, e.g., 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salt thereof, structurally related compounds, or metabolites, blended in microcrystalline cellulose, such as Avicel® brand from FMC corporation, which results in greater than 75% dissolution of the active ingredient in less than 0.25 hours in a 0.1 N HCl solution.

As used herein, the terms “sustained release”, “sustained release formulation”, “sustained release dosage formulation” and the like refer to formulations that contain materials that retard the release of active compound from the formulation relative to an “immediate release” formulation as described above, e.g., relative to an otherwise identical formulation lacking the release rate controlling polymer or other release-retarding materials. Thus, the term “sustained release” can apply to any number of extended release forms and will be considered substantially synonymous with delayed release, time release, prolonged release, time programmed release, time released, time coated release, sustained release, slow acting, long acting, delayed acting, spaced release, time spaced release, extended acting, extended action, and the like.

The terms “slow release”, “medium release” and “fast release” are intended to refer to sustained release formulations as described herein that release active compound at a rate that is slow, medium or fast rate relative to each other.

It will be appreciated that sustained release formulations can result in a release of active compound from the dosage form at a rate effective to increase the time it takes to reach maximum therapeutic concentration as compared to an immediate release formulation, for example and not limitation, by a period of 50% or more, 100% or more, 150% or more, or 200% or more as compared to an immediate release formulation; e.g., as compared to an otherwise identical formulation lacking the release rate controlling polymer or other release-retarding materials. Sustained release formulations can also result in release of active compound from the dosage form at a rate effective to decrease the maximal therapeutic concentration of said compound compared to an immediate release formulation, for example and not limitation, by at least 10%, at least 20%, at least 25%, at least 30%, at least 40%, or at least 50% compared to an immediate release formulation. Sustained release formulations can also result in release of active compound from the dosage form at a rate effective to increase the amount of time a pharmaceutically effective concentration of the active compound is maintained relative to an immediate release formulation, for example and not limitation, by at least 25%, at least 50%, at least 75%, at least 100%, or at least 125% the amount of time a pharmaceutically effective concentration of active compound is maintained relative to an immediate release formulation. Satisfaction of any of the preceding criteria is sufficient to make a formulation a “sustained release” formulation.

The present invention provides methods for sustained release of the active ingredient comprising administering to a subject the disclosed dosage forms. In one aspect, the release rate of the active compound from the dosage forms is zero order. In another aspect, the release rate of the active ingredient from the dosage forms is ascending.

As used herein, the term “release rate controlling polymer” is intended to denote any polymer material suitable for pharmaceutical dosage forms that retards the release of drug substances from such dosage forms. The release rate controlling polymer will preferably inhibit the release of the drug in the stomach. Preferably, the release rate controlling polymer will be a hydrogel that imbibes and/or absorbs fluid thereby preventing the release of the drug in the stomach. Examples of suitable release rate controlling polymers can be found in Remington's Pharmaceutical Sciences, 18th Ed., Gennaro, ed., Mack Publishing Co., Easton, Pa., 1990.

Some preferred release rate controlling polymers suitable for use in the present invention include, without limitation, hydroxypropyl celluloses, methylcelluloses, polymethacrylates, methacrylic acid-methacrylic acid ester copolymers, cellulose acetate phthalate, ethyl celluloses, hydroxyethyl celluloses, hydroxymethyl celluloses, hydroxypropylethyl celluloses, polyvinyl acetate-phthalate, hydroxypropylmethylcellulose phthalate, poly(ethylene) oxides, hydroxypropyl methyl celluloses such as, for example, hypromellose 2208 and 2910 and combinations of two or more thereof. Suitable release rate controlling polymers are available from commercial sources, such as Methocel™ K4M, Methocel™ K15M, Methocel™ K100M, Methocel™ E4M, Methocel™ K100LV, Methocel™ E50LV, Methocel™ E5, Methocel™ E6, Methocel™ E15LV, and Surelease™ available from Colorcon and Eudragit™, RS Eudragit™ RL available from Rohm GmbH & Co. In some embodiments, the formulations of the present invention will comprise high density matrix-forming hydroxypropyl methylcellulose, low density matrix-forming hydroxypropyl methylcellulose, or combinations thereof.

It will be appreciated that the different release rate controlling polymers confer different release rate properties to the formulation. By varying the type and amount of such polymers in the formulation, a wide variety of release profiles of active compound can be achieved. Those skilled in the art are able to select appropriate polymers in appropriate amounts to achieve desired release rates of active compound.

The sustained release formulations of the present invention comprise at least one release rate controlling polymer. The range of release rate controlling polymer in the formulation is preferably from about 10% to about 75% by weight, more preferably from about 20% to about 60% by weight. In one embodiment of the present invention, the amount of release rate controlling polymer in a 250 mg dosage form is from about 50 to about 150 mg. In some embodiments, the release rate controlling polymer is a cellulose ether, such as, for example, matrix-forming hydroxypropyl methylcellulose, hydoxypropyl cellulose, or hydroxyethyl cellulose, e.g., Methocel™ K4M Premium CR or Methocel™ K100M Premium CR.

In addition to comprising at least one release rate controlling polymer, sustained release dosage forms of the present invention generally comprise at least one organic acid. For uses herein, the term “organic acid” encompasses any acid that can be safely ingested by a mammal. While not wishing to be bound by any particular theory, the acid is believed to improve the release of the drug product in the intestine. Examples of organic acids suitable for use in the present invention include, but are not limited to, tartaric acid, malic acid, fumaric acid, aspartic acid, glutamic acid, glycine hydrochloride, adipic acid, succinic acid, ascorbic acid, oleic acid or citric acid. Preferred organic acids are citric acid or polyfunctional organic acid. The range of organic acid in the formulation is preferably from about 1% to about 30%, more preferably from about 2% to about 10% by weight. In one embodiment of the present invention, the amount of organic acid in a 250 mg dosage form is from about 5 to about 50 mg, preferably from about 5 to about 25 mg. In some embodiments, the amount of organic acid is from about 2 to about 50 mg.

Preferably, the sustained release formulations will be substantially free of base. For use herein, a formulation, dosage form, or composition that is substantially of base refers to a formulation, dosage form, or composition that has less than about 10% base, preferably less than about 5% base, and more preferably less than about 1% or 0.1% base. As used herein, the term “base” refers to a chemical compound that functions as a proton acceptor.

In addition to the active compound and release rate controlling polymer, the formulations of the invention can comprise any of a variety of additional materials that confer beneficial properties to the formulation. Such materials include, for example, solubility modifiers such as surfactants such as, for example, sodium lauryl sulfate, acidic compounds, antioxidants, pH modifiers, chelating agents, fillers, disentegrants, binders, lubricants, stabilizers, excipients including water soluble excipients such as sugars and water dispersing excipients such as, for example, microcrystalline cellulose, colloidal silicone dioxide, silicified microcrystalline cellulose and starch. In some embodiments, the formulation is provided at a pH of about 6 or lower, for example at a pH of from about 1 to about 6.

Nonlimiting examples of water soluble excipients or water dispersing excipients include lactose, mannitol, sucrose, and the like. The water soluble excipients can be present in a range on weight percentages depending upon the particular therapeutic objective required. For use in the present invention, percentages and parts are expressed as part by weight or percentage by weight, unless otherwise noted. In general, the range of water soluble excipients can be, for example, from about 0% to about 50% or to about 99%, or from about 2% to about 25%. Examples of water dispersible excipients include microcrystalline cellulose, colloidal silicone dioxide, silicified microcrystalline cellulose (Prosolv™), starches, croscarmelose sodium and the like.

Nonlimiting examples of stabilizers include antioxidants such as BHA, BHT, ascorbic acids, tocopherols, and the like. Nonlimiting examples of suitable metal chelators include EDTA, citric acid and the like. Nonlimiting examples of pH modifiers include citric acid, fumaric acid, and the like. Nonlimiting examples of binders include starches, PVP (polyvinylpyrrolidone), HPMC (hydroxypropyl methyl celluloses), HPC (hydroxypropyl cellulose) and the like. Nonlimiting examples of flow aids include magnesium stearate and the like. Nonlimiting examples of solubility modifiers include surfactants like sodium lauryl sulfate or polysorbate (e.g., Tween™ 80), and the like.

In a preferred embodiment, the sustained release formulations of the present invention comprise the active ingredient, at least one release rate controlling polymer, an organic acid, at least one filler and at least one lubricant.

Examples of lubricants include, but are not limited to, stearic acid, magnesium stearate, glyceryl behenate, talc, mineral oil (in PEG), colloidal silicon dioxide and the like. It will be appreciated however that any lubricant known in the art can be used in the formulations described herein. The range of lubricant can be, for example, from about 0.2% to about 5%, by weight. In one embodiment of the present invention, the amount of lubricant in a 250 mg dosage form is about 1 mg.

Examples of fillers include, but are not limited to, silicified microcrystalline cellulose, microcrystalline cellulose, cellulose acetate, cellulose diacetate, cellulose triacetate, lactose monohydrate, lactose anhydrous, calcium carbonate, calcium phosphate (e.g., dibasic anhydrous), maltodextrin, dextrose, fructose, maltose, mannitol, starch, starch (e.g., preeglatinized), sucrose, and lactose. It will be appreciated however that any filler known in the art can be used in the formulations described herein. The range of filler can be, for example, from about 25% to about 75%, or to about 99% by weight. In one embodiment of the present invention (e.g., for exemplary sustained release formulations), the amount of filler present in a 250 mg dosage form is from about 85 to about 179 mg.

The sustained release dosage forms of the present invention can comprise the active compound in any convenient percentage and part in relation to the other ingredients. Typically, the formulation comprises active ingredient in percentage of from about 0.3% to about 25%, preferably from about 0.3% to about 15%. In some embodiments, the formulation will comprise active ingredient in percentage of from about 1% to about 25%, preferably from about 2% to about 15%.

For example, in one embodiment, fast sustained release formulations comprise about 10 parts of release rate controlling polymer, and about 5 parts of organic acid per part of active ingredient, e.g., 4-cyano-N-{(2R)-2-[4-(2,3-dihydro-benzo[1,4]dioxin-5yl)-piperazin-1-yl]-propyl}-N-pyridin-2-yl-benzamide or pharmaceutically acceptable salt thereof.

In another embodiment, medium sustained release formulations comprise about 25 parts of release rate controlling polymer, and about 5 parts of organic acid per part of active ingredient, e.g., 4-cyano-N-{(2R)-2-[4-(2,3-dihydro-benzo[1,4]dioxin-5yl)-piperazin-1]-propyl}-N-pyridin-2-yl-benzamide or pharmaceutically acceptable salt thereof.

In another embodiment, slow sustained release formulations comprise about 30 parts of release rate controlling polymer, and about 1 part of organic acid per part of active ingredient, e.g., 4-cyano-N-{(2R)-2-[4-(2,3-dihydro-benzo[1,4]dioxin-5yl)-piperazin-1]-propyl}-N-pyridin-2-yl-benzamide or pharmaceutically acceptable salt thereof.

In another embodiment, sustained release formulations comprise about 18 parts of release rate controlling polymer, and about 1 part of organic acid per part of active ingredient, e.g., 4-cyano-N-{(2R)-2-[4-(2,3-dihydro-benzo[1,4]dioxin-5yl)-piperazin-1-yl]-propyl}-N-pyridin-2-yl-benzamide or pharmaceutically acceptable salt thereof.

In another embodiment, sustained release formulations comprise about 46 parts of release rate controlling polymer, and about 1 part of organic acid per part of active ingredient, e.g., 4-cyano-N-{(2R)-2-[4-(2,3-dihydro-benzo[1,4]dioxin-5yl)-piperazin-1-yl]-propyl}-N-pyridin-2-yl-benzamide or pharmaceutically acceptable salt thereof.

In some embodiments, sustained release formulations comprise about 5 mg of active ingredient, from about 50 to 150 mg of release rate controlling polymer, from about 5 to about 50 mg of organic acid, from about 85 to about 179 mg of filler and about 1 mg of lubricant.

In some embodiments, sustained release formulations comprise about 2 mg of active ingredient, from about 50 to 150 mg of release rate controlling polymer, from about 2 to about 50 mg of organic acid, from about 85 to about 179 mg of filler and about 1 mg of lubricant.

In some embodiments, exemplary sustained release formulations comprise, in a 250 mg tablet, about 5 mg of active ingredient and about 50 mg of release rate controlling polymer. Such an exemplary formulation can further comprise, for example, about 169 mg of filler, about 25 mg of organic acid (or other agent to improve release rate in the intestine) and about 1 mg of lubricant.

In some embodiments, exemplary sustained release formulations comprise, in a 250 mg tablet, about 5 mg of active ingredient and about 125 mg of release rate controlling polymer. Such an exemplary formulation can further comprise, for example, about 94 mg of filler, about 25 mg of organic acid (or other agent to improve release rate in the intestine) and about 1 mg of lubricant.

In some embodiments, exemplary sustained release formulations comprise, in a 250 mg tablet, about 5 mg of active ingredient and about 150 mg of release rate controlling polymer. Such an exemplary formulation can further comprise, for example, about 89 mg of filler, about 5 mg of organic acid (or other agent to improve release rate in the intestine) and about 1 mg of lubricant.

In some embodiments, exemplary sustained release formulations comprise, in a 250 mg tablet, about 5 mg of active ingredient and about 92 mg of release rate controlling polymer. Such an exemplary formulation can further comprise, for example, about 150 mg of filler, about 5 mg of organic acid (or other agent to improve release rate in the intestine) and about 1 mg of lubricant.

In some embodiments, exemplary sustained release formulations comprise, in a 250 mg tablet, about 2 mg of active ingredient and about 92 mg of release rate controlling polymer. Such an exemplary formulation can further comprise, for example, about 150 mg of filler, about 2 mg of organic acid (or other agent to improve release rate in the intestine) and about 1 mg of lubricant.

The sustained release formulations contemplated by the present invention can be in any form suitable for administration to a mammal and are not limited to the examples presented herein.

In some embodiments, the formulations of the invention are in the form of coated pellets or spheres. One nonlimiting example of such a formulation is a sphere containing a core of active compound in an inert matrix, coated with a release rate controlling polymer as disclosed herein. Nonlimiting examples of suitable release rate controlling polymers are pH dependent or independent polymers described herein, such as polymethacrylates, Eudragit™ IVS, Eudragit™ RS/RL, cellulose acetate phthalate, ethyl celluloses, hydroxypropyl methyl celluloses, hydroxypropyl celluloses, hydroxypropyl ethyl celluloses and the like.

In some embodiments, the formulations of the invention are in the form of pellets. Examples of such formulations include those containing pellets that contain a layer of active compound on top of an inert core, for example a sugar sphere, and a surface coating containing one or more release rate controlling polymers. In other embodiments, the formulation are in the form of capsules, e.g., hard or soft gelatin capsules and/or powder.

In some embodiments, the formulations of the invention are in the form of tablets. The percentage by weight of active compound in the representative formulations of this type is from about 0.3% to about 25%, preferably from about 0.3% to about 15%. In some embodiments, the percentage by weight of active compound in the representative formulations of this type will be about 1% to about 25%, preferably from about 2% to about 15%. Nonlimiting examples of such tablets are co-compressed tablets , e.g., “tablet-in-tablet” and matrix tablets.

The co-compressed tablet can include a core and-an-outer compressed coat. Either or both of the core and the outer compressed coat can contain active compound and/or one or more release rate controlling polymers. In some embodiments, the dosage form is a co-compressed tablet wherein both the core and the outer compressed coat contain active compound, and at least one release rate controlling polymer, one of which is preferably a hydroxypropyl methyl cellulose. Preferred matrix forming polymers include a hydroxypropyl methylcellulose selected from Methocel™ K4M, Methocel™ K15M, Methocel™ KI00M, Methocel™ E10M, Methocel™ E10M, Methocel™ E4M, Methocel K4M, Methocel™ K100LV, Methocel E50LV, Methocel™ E5, Methocel™ E6, Methocel™ E15LV or a combination of two or more thereof.

In some embodiments, the tablet is a matrix tablet. The matrix forming composition can contain waxes, gums, polyethylene oxides, carbapols, hydroxypropyl methylcelluloses, hydroxypropyl celluloses, hydroxyethyl celluloses, polymethacrylates or other release rate controlling polymers as described herein. In some embodiments, such matrix tablets are prepared by blending the active compound and the matrix forming polymer together, and compressing the blend.

In some embodiments, the tablet is a matrix tablet that includes a wax matrix. Such tablets can be prepared, for example, by melting a wax such as carnauba wax, cetostearyl alcohol or fatty acids, or combinations thereof, and adding active compound along with a filler such as microcrystalline cellulose as well as other excipients, fillers, lubricants and the like, and allowing the mixture to cool. The formulations prepared can be optionally coated with or contain one or more water soluble or release rate controlling control release polymers. The wax can be present in the formulation in a total amount by weight of, for example, from about 10% to about 60%, preferably from about 20% to about 40%. A wide variety of suitable waxes are amenable to the present invention. Nonlimiting examples of such waxes include camauba wax, cetostearyl alcohol, fatty acids, or a mixture or two or more thereof The matrix tablet also can contain one or more release rate-controlling polymers as described herein.

In some embodiments, the matrix tablet is a tablet that includes a polyethylene oxide matrix, for example and not limitation, polyethylene oxide resins such as SENTRY POLYOX™ (Union Carbide Corp.) or equivalents. Suitable POLYOX's include POLYOX™ WSR N-10, N-60 K, WSR-1105N, or WSR 303. The POLYOX™ can have a molecular weight in the range of, for example, 100,000 to 7,000,000 or 900,000 to 5,000,000. The polyethylene oxide can be present in the formulation in a total amount by weight of, for example, from about 5% or about 10% to about 40%, or about 75% preferably from about 5% to about 40% or from about 10% to about 20% of the formulation. The matrix tablet also can contain one or more release rate-controlling polymers as described herein.

In some embodiments, the matrix tablet is a tablet that includes one or more release rate controlling polymers as described herein as the matrix forming polymer. In some embodiments, such tablets include one or more matrix forming hydroxypropyl methyl celluloses as described herein as the matrix forming polymer. In some preferred embodiments, it is advantageous to use a high viscosity hydroxypropyl methylcellulose such as Methocel™ K4M at an amount by weight of, for example, from about 15% to about 70%, preferably from about 18% to about 50%. Other high viscosity polymers can also be used such as, for example, Methocel™ K15M, Methocel™ K100M, or Methocel™ E4M and the like. In some embodiments, a low viscosity hydroxypropyl methylcellulose can be used, such as Methocel™ E50LV, Methocel™ E5, Methocel™ E6, or Methocel™ E15LV or combinations thereof and the like. In certain embodiments, both a high viscosity and a low viscosity hydroxypropyl methylcellulose can be used in the matrix. In some embodiments, when the low density hydroxypropyl methylcelluloses is present in a range of from about 15% to about 70%, preferably from about 25% to about 50%, the high density hydroxypropyl methylcellulose is present in an amount by weight of from about 20% to about 50%.

In general, the active compound or ingredient can be contained within any layer of a dosage form of the invention, and sustained release of the active compound can be achieved by the use of a release rate controlling polymer either contained within the layer containing the active compound, or in any layer encompassing the layer containing the active compound, for example an enteric coating. Such an enteric coating can also be applied to pellets, beads or spheroids containing active compound, or the active compound can be contained within the enteric coating itself.

In some embodiments of the matrix tablet formulations of the invention, the active compound is present in an amount by weight of from about 0.02% to about 16%, preferably from about 0.02% to about 4%.

Tablets of the invention can be coated with water soluble film coat(s), coloring agents, or coated with pH dependent or pH independent polymers to further control the rate of release of active compound. In some embodiments, the tablets are coated with a subcoat, an enteric coating or an overcoating, or any combination thereof. In some preferred embodiments, the tablets of the formulations of the invention are coated with film.

The present inventions provides methods and/or processes for preparing sustained-release formulations comprising 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salts thereof, structurally related compounds, and/or metabolites. In one embodiment, a composition comprising the active ingredient with at least one rate controlling polymer and at least one organic acid is compressed for a time and under conditions effective for forming a tablet thereof. In some embodiments, the tablet is further coated, e.g., with film.

In another embodiment, the active ingredient is mixed with at least one release rate controlling polymer and a least one organic acid thereby forming a blend. The blend can be further compressed for a time and under conditions to form a tablet. In some embodiments, the tablet is further coated, e.g., with film. In a preferred embodiment, the blend is a dry blend.

In some embodiments, the formulations are prepared by roller compaction. For example, tablets can be prepared by granulation followed by milling. In some embodiments, the active ingredient, filler (e.g., microcrystalline cellulose) and polymer (e.g., hydroxypropylmethylcellulose) are granulated and then milled. The milled granules are then mixed with additional excipients, such as, for example, citric acid and magnesium stearate.

Also included in accordance with the present invention are any of the numerous technologies that exist for attaining sustained release oral formulations including those described above, as well as micro and macroencapsulation, fibers, matrices both polymeric (high density and low density) and non-polymeric, foams, liposomes, micelles, gels, physically dispersed drug in polymeric, porous, slightly porous or non-porous matrices, adsorption onto ion exchange resins, mixing with or adsorption onto chemically or biologically degradable matrices and the like. The active compound can be formulated in such a way that the drug achieves a single maximal concentration or can be formulated so that the drug is pulsed in two or more peaks. Oral delivery can be via way of liquid or solid dosage form. Liquid dosage forms include syrups, suspensions, emulsions, elixirs and the like. The liquid carrier can include an organic or aqueous base and can be further modified with suitable pharmaceutical additives such as solubilizers, emulsifiers, buffers, preservatives, sweeteners, flavoring agents, suspending agents, thickening agents, colorsviscosity regulators, stabilizers or osmoregulators, or combinations thereof. The aqueous carrier can also contain, for example, polymeric substances or oils.

The present invention also provides immediate release dosage forms. Immediate release dosage forms of the present invention can comprise the active ingredient, for example, 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salts thereof, structurally related compounds, or metabolites. As in the sustained release formulations, in some embodiments, the active ingredient is micronized. Preferably the immediate release formulations will be substantially free of base.

In a preferred embodiment, the immediate release formulation comprises the active ingredient, at least one filler and at least one lubricant. The formulations of the invention additionally can include any of a variety of materials that confer beneficial properties to the formulation. Such materials include, for example, solubility modifiers such as surfactants such as, for example, sodium lauryl sulfate, acidic compounds, fillers, lubricants, antioxidants, pH modifiers, chelating agents, disintegrants, binders, stabilizers, excipients including water soluble excipients such as sugars, and water dispersing excipients such as microcrystalline cellulose, colloidal silicone dioxide, silicified microcrystalline cellulose and starch. The range of lubricant is typically from about, for example, 0.2% to about 5% by weight. In one embodiment of the present invention, the amount of lubricant in a 150 mg dosage form is from about 0.5 to about 1 mg. The range of filler can be, for example, from about 70% to about 99%, by weight. In one embodiment of the present invention, the amount of filler in a 150 mg dosage form is from about 80 to about 149 mg.

The immediate release dosage forms of the present invention can contain the active compound in any convenient percentage and part in relation to the other ingredients. Typically, the formulation comprises active ingredient in percentage of from about 0.05% to about 10%.

For example, in one embodiment, immediate release formulations comprise about 297 parts of filler, and about 1.5 parts of lubricant per part of active ingredient, e.g., 4-cyano-N-{(2R)-2-[4-(2,3-dihydro-benzo[1,4]dioxin-5yl)-piperazin-1-yl]-propyl}-N-pyridin-2-yl-benzamide or pharmaceutically acceptable salt thereof

In another embodiment, immediate release formulations comprise about 29 parts of filler, and about 0.15 parts of lubricant per part of active ingredient, e.g., 4-cyano-N-{(2R)-2-[4-(2,3-dihydro-benzo[1,4]dioxin-5yl)-piperazin-1-yl]-propyl}-N-pyridin-2-yl-benzamide or pharmaceutically acceptable salt thereof.

In another embodiment, immediate release formulations comprise about 148 parts of filler, and about 0.75 parts of lubricant per part of active ingredient, e.g., 4-cyano-N-{(2R)-2-[4-(2,3-dihydro-benzo[1,4]dioxin-5yl)-piperazin-1-yl]-propyl}-N-pyridin-2-yl-benzamide or pharmaceutically acceptable salt thereof.

In another embodiment, immediate release formulations comprise about 58 parts of filler, and about 0.3 parts of lubricant per part of active ingredient, e.g., 4-cyano-N-{(2R)-2-[4-(2,3-dihydro-benzo[1,4]dioxin-5yl)-piperazin-1-yl]-propyl}-N-pyridin-2-yl-benzamide or pharmaceutically acceptable salt thereof.

The immediate release formulations contemplated by the present invention can be in any form suitable for administration to a mammal and are not limited to the examples presented herein.

The present invention provides methods and/or processes for preparing immediate release formulations comprising 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salts thereof, and/or metabolites thereof. In one embodiment, a composition comprising the active ingredient with at least one filler and at least one lubricant is compressed for a time and under conditions effective to form a tablet thereof. In some embodiments, the tablet is further coated, e.g., with film.

In some embodiments, the active ingredient is mixed with at least filler and a least one lubricant thereby forming a blend. The blend can be further compressed for a time and under conditions to form a tablet. In some embodiments, the tablet is further coated, e.g., with film.

In some embodiments, the formulations are prepared by roller compaction.

The immediate release dosage forms like the sustained release dosage forms can be, for example, in the form of coated pellets, spheres, capsules, powder, or tablets

Thus, in accordance with the present invention there are provided sustained release and immediate release dosage forms, including oral and non-oral sustained release dosage formulations. Accordingly, the present invention includes each of the numerous technologies that exist for immediate release non-oral dosage formulations. Delivery of active compound in accordance with the present invention can be via mucosal, vaginal, rectal, ocular, transdermal, intrauterine, routes and the like.

The present invention therefore provides, inter alia, dosage forms for 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salts thereof, structurally related compounds, and/or metabolites, methods for immediate delivery of 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salts thereof, structurally related compounds, and/or metabolites, and methods for sustained delivery of 4-cyano-N-{(2R)-2-[4-(2,3-dihydro-benzo[1,4]dioxin-5yl)-piperazin-1-yl]-propyl}-N-pyridin-2-yl-benzamide, pharmaceutically acceptable salts thereof, structurally related compounds, and/or metabolites over an extended period of time. In some embodiments, administration of the dosage form will be once every 24 hours, once every 12 hours, or once every 6 hours.

In some embodiments, the active ingredient is released at a uniform release rate. By “uniform release rate” is meant an average hourly release rate from the core that varies positively or negatively by no more than about 30% and preferably no more than about 25% and most preferably no more than 10% from either the preceding or the subsequent average hourly release rate.

In some embodiment, the active ingredient is released in a prolonged period of time. By “prolonged period of time” is meant a continuous period of time of at least about 4 hours, preferably 6-8 hours or more and, more preferably, 10 hours, 15 hours or more. For example, in some embodiments, the sustained release dosages forms described herein begin releasing therapeutic agent at a uniform release rate within about 1 to about 6 hours, or about 2 to about 6 hours following administration and the uniform rate of release, as defined above, continues for a prolonged period of time from about 25% to until at least about 75% and preferably at least about 85% of the drug is released from the dosage form. Release of therapeutic agent continues thereafter for several more hours although the rate of release is generally slowed somewhat from the uniform release rate.

In some embodiments, the dosage form is formulated to release the active ingredient at a rate that is effective to achieve a maximal plasma concentration at about 1 to about 12 hours following administration. In some embodiments, the dosage form will be formulated to release the active ingredient at a rate that is effective to achieve a maximal plasma concentration at about 1 to about 4 hours following administration. In some embodiments, the dosage form will be formulated to release the active ingredient at a rate that is effective to achieve a plasma concentration this is about 50% of the maximal plasma concentration at about 15 hours following administration, preferably at about 1 to about 10 hours following administration.

In other embodiments, dosage form is formulated to release the active ingredient at a rate that is effective to achieve a maximal plasma concentration at about 6 or about 12 hours following administration.

The “plasma drug concentration” or “plasma concentration” refers to the concentration of drug in the blood plasma of a subject, generally expressed as mass per unit volume, typically nanograms per milliliter. The plasma drug concentration at any time following drug administration is referenced as Cₜᵢₘₑ, as in C₉ₕ or C₂₄ₕ.

Persons of skill in the art appreciate that plasma drug concentrations obtained in individual subjects will vary due to interpatient variability in the many parameters affecting drug absorption, distribution, metabolism and excretion. For this reason, unless otherwise indicated, mean values obtained from groups of subjects are used herein for purposes of comparing plasma drug concentration data and for analyzing relationships between in vitro dosage form dissolution rates and in vivo plasma drug concentrations.

The dosage formulations described herein facilitate the immediate or sustained release of active compounds in a mammal through many routes, including oral administration. In some preferred embodiments, the formulations include the compound 4-cyano-N-{(2R)-2-[4-(2,3-dihydro-benzo[1,4]dioxin-5yl)-piperazin-1-yl]-propyl}-N-pyridin-2-yl-benzamide, preferably the hydrochloride salt thereof.

### EXAMPLES

### Example 1

### Identification of metabolites of 4-cyano-N-{(2R)-2-[4-(2,3-dihydro-benzo[1,4]dioxin-5yl)-piperazin-1-yl]-propyl}-N-pyridin-2-yl-benzamide

Four metabolites referred to as M8, M11, M12, and M13 were isolated by solvent (ethyl acetate containing 10% methano) extraction followed by semi-preparative HPLC. The semi-preparative HPLC separation was conducted on a xTerra C18 column (7.8×300 mm, 10 μm), and a gradient of acetonitrile/water containing 10 mM ammonium acetate (pH=4.5) was used as the mobile phase.

The structures of the metabolites were determined based on NMR and mass spectra data. For NMR, all samples were dissolved in CD₃CN. For sample M11, about 10% D₂O was added to increase solubility. Proton and COSY data were acquired on all samples. For the samples containing M11 and M12, HSQC and HMBC data were also acquired to determine the structures.

M11, M12 and M13 metabolites were formed through hydroxylation at the dihydrobenzo-[1,4]dioxin-piperazine moiety. The NMR studies were conducted to determine the locations of the hydroxylation in these metabolites and to confirm the structure of M8.

M11: 1D proton spectrum of M11 showed two aromatic protons of the 2,3-dihydro-benzo[1,4]dixon moiety instead of 3 as in the parent compound, indicating the hydroxylation occurred on the benzene ring. The two proton signals are doublets suggesting the hydroxylation occurred either on C₆ or C₈ positions. To distinguish the two regio isomers, a ID NOE experiment was carried out, since relatively strong NOE correlations between the benzene proton H₆ and the piperazine protons are expected for C8 hydroxylation, but not for C₆ hydroxylation. Such NOE correlations were indeed observed in the ID NOE experiment. Therefore the structure of M11 is as shown below where the hydroxyl group is on C₈ of the 2,3-dihydro-benzo[1,4]dixon moiety.

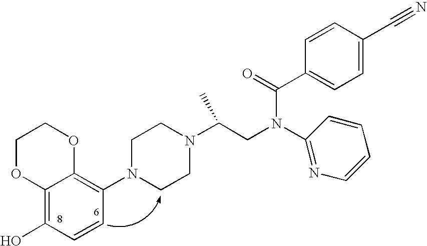

M12: ID proton spectrum of M12 was more complicated than expected for the metabolite. A careful analysis of the spectrum however, suggested the sample contained isomers. Comparison of the proton spectrum of the M12 metabolite with that of the parent compound indicated that the aromatic moieties and the piperazin moiety are intact in M12. The protons of the 1,4-dioxin ring however, are quite different. Three methine signals are observed at 5.5, 5.15 sand 5.1 ppm. These methine protons integrated into one equivalent proton for the sample. The HSQC data showed that the carbon shifts of these methine groups are between 80 to 88 ppm, suggesting the hydroxylation on one of the dioxin methylenes. COSY spectrum showed that the down-filed methine proton correlates to the methylene protons of the dioxane ring, confirming the hydroxylation on the dioxane ring. The fact that more than two sets of signals were observed indicates chiral isomers existed in the sample. Whether the chiral isomers were generated by the enzymes or through racemization in the sample purification steps is not clear. Based on the NMR results the structure of M12 is as shown below:

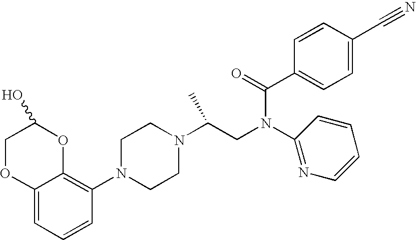

M13: Comparison of the proton spectrum of M13 with that of M12 suggests M12 and M13 are very similar. All aromatic protons observed in the parent compound were observed in M13 suggesting that the aromatic moieties are intact in the metabolite. It appeared that in M13, the hydroxylation also occurred on the dioxin ring. Similar to M12, M13 contained isomers as indicated by four methine protons observed at 5.5, 5.19, 5.10 and 4.86 ppm. It was noted that over time the intensity of these four methine signals changed, suggesting the ratio of the isomers have changed. Similar changes were observed in M12. Combined with the results from M12 analysis, it appeared that the observed NMR spectra of M12 and M13 might not represent the original components. The NMR analysis indicated that M12 and M13 were produced by hydroxylation on the dioxane ring, corresponding to 2 and 3 positions, respectively. M12 and M13 can rearrange, and both can be racemized.

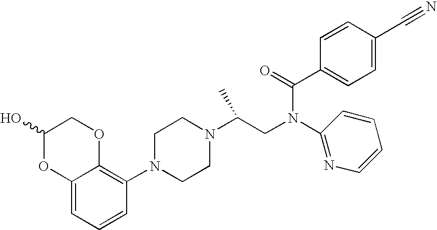

M8: Proton and COSY spectra of M8 were acquired for this sample. The data are consistent with the proposed structure for M8 based on MS/MS analysis performed by DSM. The pyridine moiety, the piperazin moiety and the cyano-propyl benzamide moieties are all intact. Compared with the parent compound, the only group missing is the 2,3-dihydro-benzo[1,4]dioxin moiety.

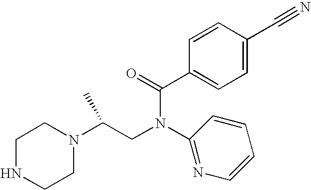

### Example 2

### Identification of compounds structurally related to 4-cyano-N-{(2R)-2-[4-(2,3-dihydro-benzo[1,4]dioxin-5yl)-piperazin-1-yl-propyl}-N-pyridin-2-yl-benzamide

Structurally related compounds represented by Formulas 2-9 were identified. The structurally related compounds were isolated from a preparation comprising 4-cyano-N-{(2R)-2-[4-(2,3-dihydro-benzo[1,4]dioxin-5yl)-piperazin-1-yl]-propyl}-N-pyridin-2-yl-benzamide by preparative chromatography in the amounts of about 1 mg with the purity of about 90%. The structures were established by nuclear magnetic resonance spectroscopy, electrospray ioniozation mass spectrometry and determination of the number of exchangeable protons.

A preparation comprising 4-cyano-N-{(2R)-2-[4-(2,3-dihydro-benzo[1,4]dioxin-5yl)-piperazin-1-yl]-propyl}-N-pyridin-2-yl-benzamide hydrochloride salt was further processed as follows. The starting material was converted to base by treatment with aqueous sodium hydroxide and ethyl acetate. The resulting ethyl acetate solution was dried azeotropically, diluted with heptane to give a 3:1 ethyl acetate heptane mixture and treated with silica gel. The resulting mixture was filtered and concentrated repeatedly to remove heptane. The base was treated in ethyl acetate solution with 1.0 equivalent of hydrogen chloride in ethyl acetate. The product was dissolved in hot denatured ethanol. The mixture was filtered and concentrated. The product was crystallized by cooling and isolated by filtration. The final wet cake was dried. This process reduced the levels of dimeric impurities of 4-cyano-N-{(2R)-2-[4-(2,3-dihydro-benzo[1,4]dioxin-5yl)-piperazin-1-yl]-propyl}-N-pyridin-2-yl-benzamide.

### Example 3

### Representative Sustained Release Formulations of the Present Invention

| | Function of | Amount per Tablet (mg) | | |
|---|---|---|---|---|
| Ingredient | Ingredient | Fast | Medium | Slow |

Active Core:

| | | | | |
|---|---|---|---|---|
| 4-cyano-N-{(2R)- | Active | 5.0 | 5.0 | 5.0 |
| 2-[4-(2,3-dihydro- | | | | |
| benzo[1,4]dioxin- | | | | |
| 5yl)-piperazin-1- | | | | |
| yl]-propyl}-N- | | | | |
| pyridin-2-yl- | | | | |
| benzamide | | | | |
| hydrochloride^{a} | | | | |
| Silicified | Filler | 169.0 | 94.0 | 88.75 |
| microcrystalline | | | | |
| cellulose | | | | |
| (ProSolv ® HD 90) | | | | |
| HPMC | Polymer | 50.0 | 125.0 | 37.5 |
| (Methocel ™ | | | | |
| K4M Premium | | | | |
| CR) | | | | |
| HPMC | Polymer | | | 112.75 |
| (Methocel ™ | | | | |
| K100M Premim | | | | |
| CR) | | | | |
| Mg Stearate NF | Lubricant | 1.0 | 1.0 | 1.0 |
| Citric Acid, | To improve | 25.0 | 25.0 | 5.0 |
| Anhydrous | release | | | |
| | Rate in | | | |
| | intestine | *** | *** | *** |
| Weight of Core | | 250 | 250 | 250 |
| (mg) | | | | |

Film Coating:

| | | | | |
|---|---|---|---|---|
| Opadry White | White Film | 7.5 | 7.5 | 7.5 |
| (YS-1- | | | | |
| 18202A) | | | | |
| Opadry Clear | Clear Film | 1.25 | 1.25 | 1.25 |
| (YS-1- | | | | |
| 19025A) | | ***.** | ***.** | ***.** |
| Total Tablet Wt. | | 258.75 | 258.75 | 258.75 |
| (mg) | | | | |

^{a}The amount of active ingredient may need to be adjusted according to its release potency.

### Example 4

### Representative Sustained Release Formulations of the Present Invention

| | | |
|---|---|---|
| | Function of | |
| Ingredient | Ingredient | Amount per Tablet (mg) |

| | | | |
|---|---|---|---|
| 4-cyano-N-{(2R)- | Active | 5.0 | 2.0 |
| 2-[4-(2,3-dihydro- | | | |
| benzo[l,4]dioxin- | | | |
| 5yl)-piperazin-1- | | | |
| yl]-propyl}-N- | | | |
| pyridin-2-yl- | | | |
| benzamide | | | |
| hydrochloride^{a} | | | |
| Microcrystalline | Filler | 46.5 | 52.5 |
| cellulose | | | |
| (Avicel ® PH112) | | | |
| Fast-Flow Lactose | Filler | 100.00 | 100.00 |
| HPMC | Polymer | 55.00 | 55.00 |
| (Methocel ™ | | | |
| K4M Premium | | | |
| CR) | | | |
| HPMC | Polymer | 37.50 | 37.50 |
| (Methocel ™ | | | |
| K100LV Premim | | | |
| CR LH) | | | |
| Mg Stearate NF | Lubricant | 1.0 | 1.0 |
| Citric Acid, | To improve | 5.00 | 2.00 |
| Anhydrous | release | | |
| | Rate in | | |
| | intestine | *** | *** |
| Weight of Core | | 250 | 250 |
| (mg) | | | |
| Opadry White | White Film | 7.5 | 7.5 |
| (YS-1- | | | |
| 18202A) | | | |
| Opadry Clear | Clear Film | 1.25 | 1.25 |
| (YS-1- | | | |
| 19025A) | | ***.** | ***.** |
| Total Tablet Wt. | | 258.75 | 258.75 |
| (mg) | | | |

a: The amount of active ingredient may need to be adjusted according to its release potency.

### Example 5

### Representative Immediate Release Formulations of the Present Invention

0.5 mg Tablets

| | Claim | | Input |
|---|---|---|---|
| Ingredient | (mg) | % Wt/Wt | (mg/tablet) |
| Active ingred. micronized^{a,b} | 0.5 | 0.33 | 0.50 |
| Lactose Monohydrate, NF^{b} | | 79.17 | 118.75 |
| Microcrystalline cellulose, NF | | 20.00 | 30.00 |
| Magnesium stearate, NF | | .50 | 0.75 |
| Total | | 100.0 | 150.00 |

The active ingredient is 4-cyano-N-{(2R)-2-[4-(2,3-dihydro-benzo[1,4]dioxin-5yl)-piperazin-1-yl]-propyl}-N-pyridin-2-yl-benzamide hydrochloride
^{a}The active moiety portion (free base) is theoretically 93% of 4-cyano-N-{(2R)-2-[4-(2,3-dihydro-benzo[1,4]dioxin-5yl)-piperazin-1-yl]-propyl}-N-pyridin-2-yl-benzamide hydrochloride drug substance. Actual amounts added are based on the potency of the hydrochloride drug substance. Inputs listed in the table above are based on the weight of the active ingredient.
^{b}If the hydrochloride drug substance is not at 100% potency, adjustment to the drug substance input must be made with corresponding adjustment to the lactose monohydrate input.
^{c}Includes an excess quantity. Theoretical quantity is 0.075 Kg.

1.0 mg Tablets,

| | Claim | | Input |
|---|---|---|---|
| Ingredient | (mg) | % Wt/Wt | (mg/tablet) |
| Active ingred. micronized^{a,b} | 1.0 | 0.67 | 1.0 |
| Lactose Monohydrate, NF^{b} | | 78.83 | 118.25 |
| Microcrystalline cellulose, NF | | 20.00 | 30.00 |
| Magnesium stearate, NF | | 0.50 | 0.75 |
| Total | | 100.0 | 150.00 |

The active ingredient is 4-cyano-N-{(2R)-2-[4-(2,3-dihydro-benzo[1,4]dioxin-5yl)-piperazin-1-yl]-propyl}-N-pyridin-2-yl-benzamide hydrochloride
^{a}The active moiety portion (free base) is theoretically 93% of 4-cyano-N-{(2R)-2-[4-(2,3-dihydro-benzo[1,4]dioxin-5yl)-piperazin-1-yl]-propyl}-N-pyridin-2-yl-benzamide hydrochloride drug substance.
Actual amounts added are based on the potency of the hydrochloride drug substance. Inputs listed in the table above are based on the weight of the active ingredient.
^{b}If the hydrochloride drug substance is not at 100% potency, adjustment to the drug substance input must be made with corresponding adjustment to the lactose monohydrate input.
^{c}Includes an excess quantity. Theoretical quantity is 0.075 Kg

2.5 mg Tablets

| | Claim | % | Input |
|---|---|---|---|
| Ingredient | (mg) | Wt/Wt | (mg/tablet) |
| Active ingred. micronized^{a,b} | 2.5 | 1.67 | 2.50 |
| Lactose Monohydrate, NF^{b} | | 77.83 | 116.75 |
| Microcrystalline cellulose, NF | | 20.00 | 30.00 |
| Magnesium stearate, NF | | .50 | 0.75 |
| Total | | 100.0 | 150.00 |

The active ingredient is 4-cyano-N-{(2R)-2-[4-(2,3-dihydro-benzo[1,4]dioxin-5yl)-piperazin-1-yl]-propyl}-N-pyridin-2-yl-benzamide hydrochloride
^{a}The active moiety portion (free base) is theoretically 93% of 4-cyano-N-{(2R)-2-[4-(2,3-dihydro-benzo[1,4]dioxin-5yl)-piperazin-1-yl]-propyl}-N-pyridin-2-yl-benzamide hydrochloride drug substance. Actual amounts added are based on the potency of the hydrochloride drug substance. Inputs listed in the table above are based on the weight of the active ingredient.
^{b}If the hydrochloride drug substance is not at 100% potency, adjustment to the drug substance input must be made with corresponding adjustment to the lactose monohydrate input.
^{c}Includes an excess quantity. Theoretical quantity is 0.075 Kg

5.0 mg Tablets

| | Claim | % | Input |
|---|---|---|---|
| Ingredient | (mg) | Wt/Wt | (mg/tablet) |
| Active ingred. micronized^{a,b} | 5.0 | 3.33 | 5.0 |
| Lactose Monohydrate, NF^{b} | | 76.17 | 114.25 |
| Microcrystalline cellulose, NF | | 20.00 | 30.00 |
| Magnesium stearate, NF | | .50 | 0.75 |
| Total | | 100.0 | 150.00 |

The active ingredient is 4-cyano-N-{(2R)-2-[4-(2,3-dihydro-benzo[1,4]dioxin-5yl)-piperazin-1-yl]-propyl}-N-pyridin-2-yl-benzamide hydrochloride
^{a}The active moiety portion (free base) is theoretically 93% of 4-cyano-N-{(2R)-2-[4-(2,3-dihydro-benzo[1,4]dioxin-5yl)-piperazin-1-yl]-propyl}-N-pyridin-2-yl-benzamide hydrochloride drug substance.
Actual amounts added are based on the potency of the hydrochloride drug substance. Inputs listed in the table above are based on the weight of the active ingredient.
^{b}If the hydrochloride drug substance is not at 100% potency, adjustment to the drug substance input must be made with corresponding adjustment to the lactose monohydrate input.
^{c}Includes an excess quantity. Theoretical quantity is 0.075 Kg

### Example 6

### Representative Manufacturing Directions for Representative Immediate Release Tablets

1. Dispense the lactose monohydrate and microcrystalline cellulose into suitable containers.

2. Dispense the 4-cyano-N-{(2R)-[4-(2,3-dihydro-benzo[1,4]dioxin-5yl)-piperazin-1-yl]-propyl}-N-pyridin-2-yl-benzamide hydrochloride into a suitably sized tumbler mixing bowl. Add a small portion of the dispensed lactose monohydrate and mix into a tumbling mixer.

3. Pass the pre-blend from step 2, followed by the microcrystalline cellulose, through a 500 μm screen into a suitably sized tumbler mixer bowl. Mix

4. Transfer the pre-blend from step 3 into a suitably sized tumbler mixer bowl. Pass the remaining lactose monohydrate through a 500 μm screen into the mixing bowl. Mix.

5. Weigh the blend and calculate the amount of magnesium stearate required for the batch. Dispense the magnesium stearate into a suitable container, and mix with a portion of the blend from step 4.

6. Pass this pre-mix through a 500 μm screen and into the remaining blend in the mixing bowl. Mix the final blend.

7. Compress the blend from step 6 using a suitable compression machine fitted with appropriate tooling, to produce tablets with the required weight and hardness.

8. De-dust, weight check and visually inspect the finished tablets.

1. A compound that is 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof in the form of particles having a mean diameter of no more than about 20 microns.

2. The compound of claim 1 in the form of particles having a mean diameter of about 0.75 to about 10 microns.

3. The compound of claim 1 in the form of particles having a mean diameter of about 2 to about 8 microns.

4. A composition comprising 4-cyano-N-{(2R)-2-[4-(2,3-dihydro-benzo[1,4]dioxin-5-yl-piperazin-1-yl]-propyl}-N-pyridin-2-yl-benzamide that is substantially free of dimers of 4-cyano-N-{(2R)-2-[4-(2,3-dihydro-benzo[1,4]dioxin-5-yl)-piperazin-1-yl]-propyl}-N-pyridin-2-yl-benzamide.

5. The composition of claim 4 comprising less than about 0.1 weight percent each of dimers of 4-cyano-N-{(2R)-2-[4-(2,3-dihydro-benzo[1,4]dioxin-5yl)-piperazin-1-yl]-propyl}-N-pyridin-2-yl-benzamide.

6. The composition of claim 4 wherein said dimers are selected from the group consisting of Formula 7 and Formula 8: 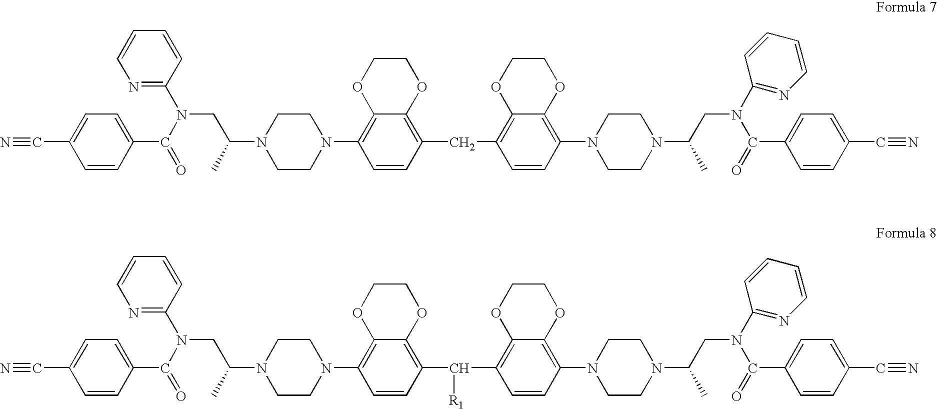
wherein R₁ is —CH₃, —CH(CH₃)₂, —CH₂CH₂CH₃, CH₂CH₂CH₂CH₃ or —CH₂CH₂CH₂CH₂CH₂.

7. A composition comprising {(2R)-2-[4-(2,3-dihydro-benzo[1,4]dioxin-5-yl)-piperazin-1-yl]-propyl}-N-pyridin-2-yl-amine or a pharmaceutically acceptable salt thereof.

8. A composition comprising 4-cyano-N-{(2S)-2-[4-(2,3-dihydro-benzo[1,4]dioxin-5-yl)-piperazin-1-yl]-propyl}-N-pyridin-2-yl-benzamide or a pharmaceutically acceptable salt thereof.

9. A composition comprising:
4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof;
{(2R)-2-[4-(2,3-dihydro-benzo[1,4]dioxin-5-yl)-piperazin-1-yl]-propyl}-N-pyridin-2-yl-amine or a pharmaceutically acceptable salt thereof; and
4-cyano-N-{(2S)-2-[4-(2,3-dihydro-benzo[1,4]dioxin-5-yl)-piperazin-1-yl]-propyl}-N-pyridin-2-yl-benzamide or a pharmaceutically acceptable salt thereof.

10. The composition of claim 9 comprising:
about 0.1 weight percent of said{(2R)-2-[4-(2,3-dihydro-benzo[1,4]dioxin-5-yl)-piperazin-1-yl]-propyl}-N-pyridin-2-yl-amine;
about 0.1 weight percent of said 4-cyano-N-{(2S)-2-[4-(2,3-dihydro-benzo[1,4]dioxin-5-yl)-piperazin-1-yl]-propyl}-N-pyridin-2-yl-benzamide hydrochloride salt; and
a remainder of said 4-cyano-N-{(2R)-2-[4-(2,3-dihydro-benzo[1,4]dioxin-5-yl)-piperazin-1-yl]-propyl}-N-pyridin-2-yl-benzamide hydrochloride salt.

11. The composition of claim 9 further comprising at least one release rate controlling polymer and at least one organic acid.

12. A dosage form comprising 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof, wherein said 4-cyano-N-{(2R)-2-[4-(2,3-dihydro-benzo[1,4]dioxin-5-yl)-piperazin-1yl ]-propyl)-N-pyridin-2-yl-benzamide or a pharmaceutically acceptable salt thereof is released at rate that is effective to achieve a maximal plasma concentration at about 1 to about 12 hours following administration.

13. The dosage form of claim 12 wherein said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof is released at rate that is effective to achieve a plasma concentration that is about 50% of said maximal plasma concentration at about 1 to about 10 hours following administration.

14. A dosage form comprising:
4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof;
at least one release rate controlling polymer; and
at least one organic acid.

15. The dosage form of claim 14 that is substantially free of dimers of 4-cyano-N-{(2R)-2-[4-(2,3-dihydro-benzo[1,4]dioxin-5-yl)-piperazin-1-yl]-propyl}-N-pyridin-2-yl-benzamide.

16. The dosage form of claim 15 comprising less than about 0.1 weight percent each of dimers of 4-cyano-N-{(2R)-2-[4-(2,3-dihydro-benzo[1,4]dioxin-5yl)-piperazin-1-yl]propyl}-N-pyridin-2-yl-benzamide.

17. The dosage form of claim 15 wherein said dimers are selected from the group consisting of Formula 7 and Formula 8 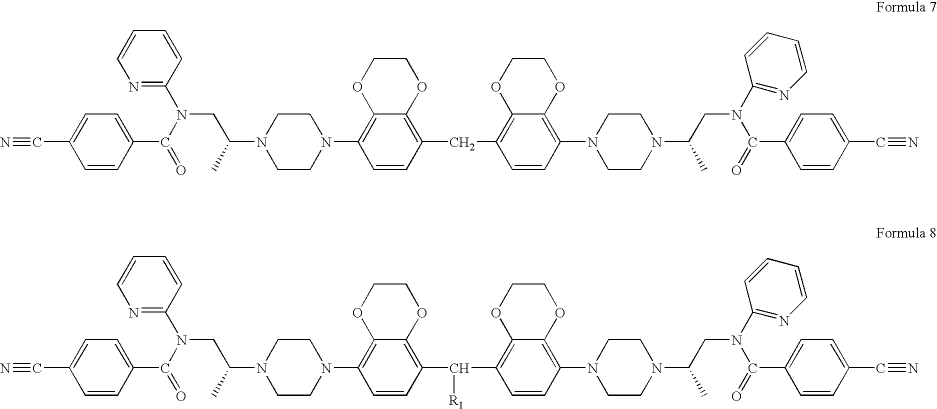
wherein R₁ is —CH₃, —CH(CH₃)₂, —CH₂CH₂CH₃, CH₂CH₂CH₂CH₃ or —CH₂CH₂CH₂CH₂CH₃.

18. The dosage form of claim 14 wherein said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof is micronized.

19. The dosage form of claim 14 wherein said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof is in the form of particles having a mean diameter of no more than about 20 microns.

20. The dosage claim 14 wherein said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof is in the form of particles having a mean diameter of about 0.75 to about 10 microns.

21. The dosage form of claim 14 wherein said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof is in the form of particles having a mean diameter of about 2 to about 8 microns

22. The dosage form of claim 14 that is substantially free of base.

23. The dosage form of claim 14 wherein said organic acid is citric acid.

24. The dosage form of claim 14 wherein said at least one release rate controlling polymer is a methylcellulose.

25. The dosage form of claim 24 wherein said at least one release rate controlling polymer is a hydroxypropyl methylcellulose or hydroxypropyl methylcellulose phthalate.

26. The dosage form of claim 25 wherein said at least one release rate controlling polymer is a hydroxypropyl methylcellulose.

27. The dosage form of claim 14 further comprising at least one filler.

28. The dosage form of claim 27 wherein said at least one filler is microcrystalline cellulose.

29. The dosage form of claim 14 further comprising at least one lubricant.

30. The dosage form of claim 29 wherein said at least one lubricant is magnesium stearate.

31. The dosage form of claim 14 that comprises about 2 to about 46 parts of said release rate controlling polymer and about 0.4 to about 10 parts of said organic acid per part of said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof.

32. The dosage form of claim 14 that comprises about 10 parts of said release rate controlling polymer and about 5 parts of said organic acid per part of said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof.

33. The dosage form of claim 14 that comprises about 25 parts of said release rate controlling polymer and about 5 parts of said organic acid per part of said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof.

34. The dosage form of claim 14 that comprises about 30 parts of said release rate controlling polymer and about 1 part of said organic acid per part of said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof.

35. The dosage form of claim 14 that comprises about 18 parts of said release rate controlling polymer and about 1 part of said organic acid per part of said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof.

36. The dosage form of claim 14 that comprises about 46 parts of said release rate controlling polymer and about 1 part of said organic acid per part of said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof.

37. The dosage form of claim 14 that comprises about 5 mg of said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof.

38. The dosage form of claim 14 that comprises about 2 mg of said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof.

39. The dosage form of claim 14 that is in the form of a tablet.

40. The dosage form of claim 14 wherein said compound is released at rate that is effective to achieve a maximal plasma concentration at about 1 to about 12 hours following administration.

41. The dosage form of claim 14 wherein said compound is released at rate that is effective to achieve a plasma concentration that is about 50% of a maximal plasma concentration at about 1 to about 10 hours following administration.

42. A composition comprising:
4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof;
at least one release rate controlling polymer; and
at least one organic acid.

43. The composition of claim 42 that is substantially free of base.

44. The composition of claim 42 that is in the form of a dry blend.

45. A dosage form comprising:
4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof;
at least one filler; and
at least one lubricant.

46. The dosage form of claim 45 wherein said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof is micronized.

47. The dosage form of claim 45 wherein said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof is in the form of particles having a mean diameter of no more than about 10 microns.

48. The dosage form of claim 45 wherein said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof is in the form of particles having a mean diameter of about 20 microns.

49. The dosage form of claim 45 wherein said 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof is in the form of particles having a mean diameter of about 0.75 to about 10 microns.

50. The dosage form of claim 45 wherein said 4-cyano-N-{(2R)-2-[4-(2,3-dihydro-benzo[1,4]dioxin-5-yl)-piperazin-1-yl]-propyl}-N-pyridin-2-yl-benzamide is present in the form of its hydrochloride salt.

51. The dosage form of claim 45 that is substantially free of base.

52. The dosage form of claim 45 wherein said at least one filler is a blend of microcrystalline cellulose and lactose.

53. The dosage form of claim 45 wherein said at least one lubricant is magnesium stearate.

54. The dosage form of claim 45 that is in the form of a tablet.

55. A composition comprising:
4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof;
at least one filler; and
at least one lubricant.

56. A compound that is 4-cyano-N-{(2R)-2-[4-(8-hydroxy-2,3-dihydro-benzo[1,4]dioxin-5-yl)-piperazin-1-yl]-propyl}-N-pyridin-2-yl-benzamide or a pharmaceutically acceptable salt thereof.

57. A compound that is 4-cyano-N-{(2R)-2-[4-(3-hydroxy-2,3-dihydro-benzo[1,4]dioxin yl)-piperazin-1-yl]-propyl}-N-pyridin-2-yl-benzamide or a pharmaceutically acceptable salt thereof.

58. A compound that 4-cyano-N-{(2R)-2-[4-(2-hydroxy-2,3-dihydro-benzo[1,4]dioxin-5piperazin-1-yl]-propyl}-N-pyridin-2-yl-benzamide or a pharmaceutically acceptable salt thereof.

59. A compound that is 4-cyano-N-(2R-2-piperazin-1-yl-propyl)-N-pyridin-2-yl-benzamide or a pharmaceutically acceptable salt thereof.
